(19) United States
(12) Patent Application Publication
(10) Pub. No.: US 2019/0321233 A1
(43) Pub. Date: Oct. 24, 2019

(54) Temporary Barrier to Shield an Exposed Feature On a Person

(71) Applicant: 2G Medical, LLC, Clearwater, FL (US)

(72) Inventors: Warren C. Wallis, Clearwater, FL (US); Neil Avery Muchin, Oakland, CA (US)

(21) Appl. No.: 16/374,263

(22) Filed: Apr. 3, 2019

Related U.S. Application Data
(60) Provisional application No. 62/660,302, filed on Apr. 20, 2018.

(51) Int. Cl. A61F 13/02; B32B 7/14; B32B 27/40

(52) U.S. Cl. CPC A61F 13/025; B32B 7/14; B32B 27/40; A61F 13/0266; A61F 13/0236; A61F 2013/00319; B32B 2307/732; B32B 2307/748; B32B 2535/00; B32B 2307/7265; B32B 2307/412

(57) ABSTRACT

In general, in an aspect, a barrier includes a water impenetrable membrane having two sides. On one side of the membrane, a continuous strip of adhesive surrounds an adhesive-free central area. The entire extent of the continuous strip of adhesive bears one or more release strips. The membrane is resilient, flexible, and stretchable with an elongation at break in the range of 150% to 1000% (using test ASTM D412).

### CROSS-REFERENCE TO RELATED APPLICATIONS

This application claims the benefit of and priority of U.S. Provisional Application Ser. No. 62/660,302, filed on Apr. 20, 2018, the entire contents of which are incorporated here by reference.

### BACKGROUND

This description relates to a temporary barrier to shield an exposed feature on a person. Exposed features can include skin, nails, eyes, or other external parts of a living body and also abnormal or other conditions such as wounds, incisions, openings, vascular access sites, dressings, abrasions, infections, or treatment areas. Water, other liquids, vapors, gases, solids, and a wide variety of other environmental materials can alter, contaminate, infect, degrade, damage, or otherwise affect exposed features. In some cases, the exposed features can be said to be vulnerable exposed features in that they may undergo unwanted changes if they come into contact with particular environmental materials. In one example, a healing wound on a person's upper forearm can become infected or damaged if it comes into contact with water during a shower.

### SUMMARY

In general, in an aspect, a barrier includes a water impenetrable membrane having two sides. On one side of the membrane, a continuous strip of adhesive surrounds an adhesive-free central area. The entire extent of the continuous strip of adhesive bears one or more release strips. The membrane is resilient, flexible, and stretchable with an elongation at break in the range of 150% to 1000% (using test ASTM D412).

Implementations may include one or a combination of two or more of the following features. The membrane includes a polymeric material. The polymeric material includes a polyurethane. The polyurethane includes an aromatic polyether polyurethane. The membrane is transparent. The membrane has a thickness in the range of 2 mils to 4 mils inches. The adhesive has adhesive properties to seal the membrane to skin temporarily and to permit removable of the membrane without damaging the skin.

In general, in an aspect, a method includes attaching a barrier to skin of a person to form a seal between the barrier and the skin continuously along a strip that surrounds a vulnerable exposed feature of the person. The barrier shields the vulnerable exposed feature temporarily against exposure to a detrimental environmental material. The barrier stretches to accommodate a tension imparted to the barrier by motion of a part of the person in the vicinity of the vulnerable exposed feature, to prevent the tension from breaking the seal.

Implementations may include one or a combination of two or more of the following features. The forming of the seal between the barrier and the skin includes pressing an adhesive material against the skin. The seal can be broken and the barrier removed from the skin. The barrier contracts when the tension imparted to the barrier is released.

These and other aspects, features, and implementations (a) can be expressed as methods, apparatus, systems, components, program products, methods of doing business, means or steps for performing a function, and in other ways and (b) will become apparent from the following description, including the claims.

### DESCRIPTION

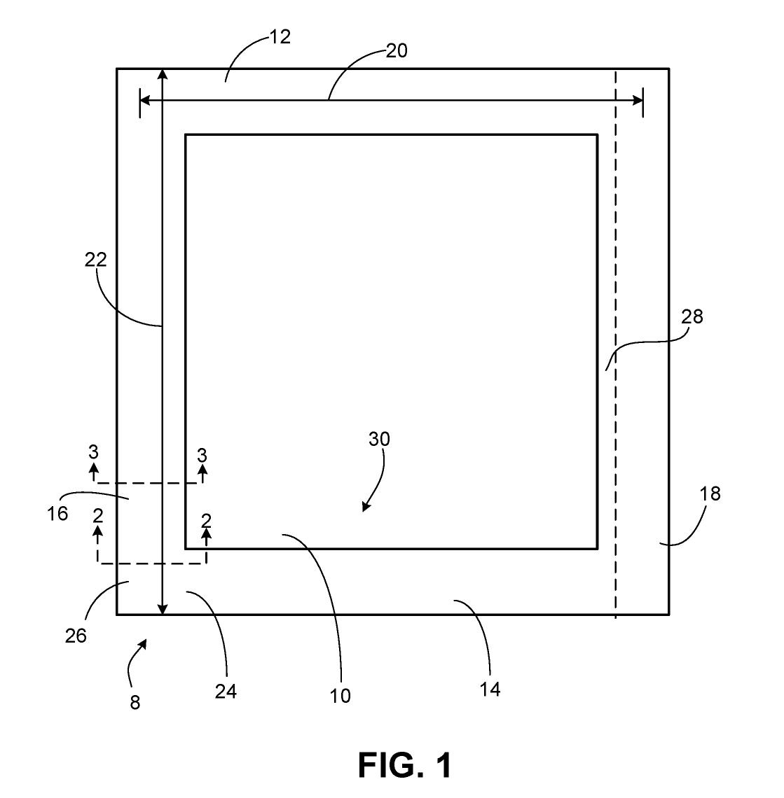

FIG. 1 is a perspective view of a barrier.

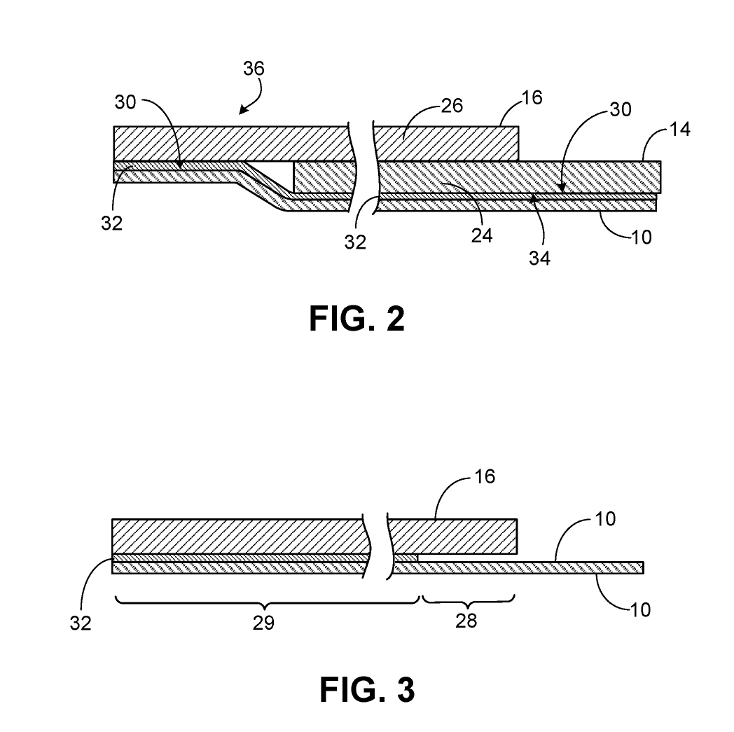

FIG. 2 is a sectional view at 2-2 of FIG. 1.

FIG. 3 is a sectional view at 3-3 of FIG. 1.

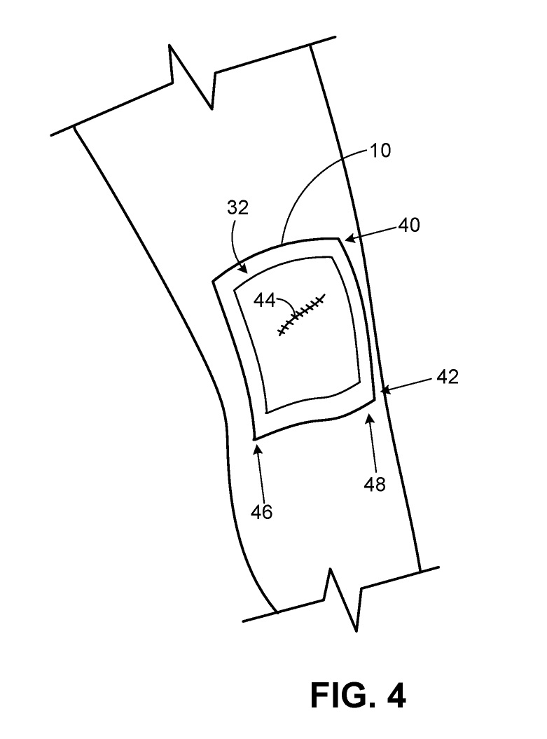

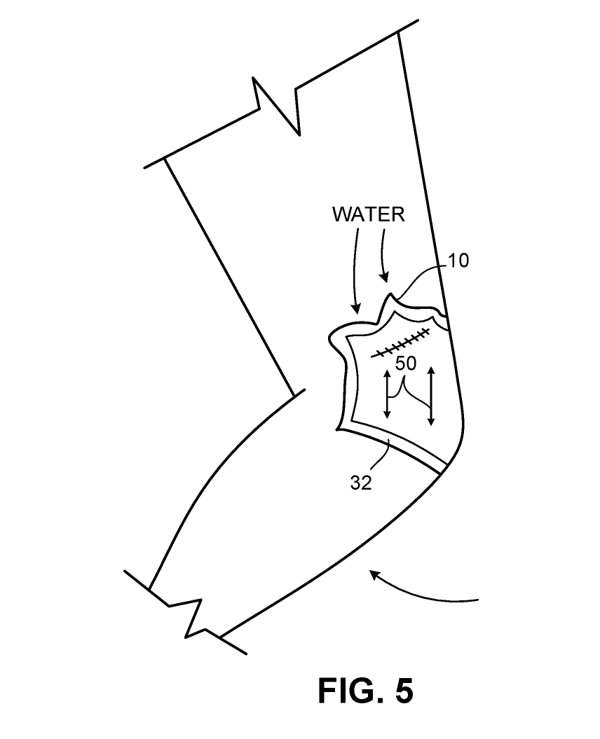

FIGS. 4 and 5 are perspective views of a limb bearing a barrier.

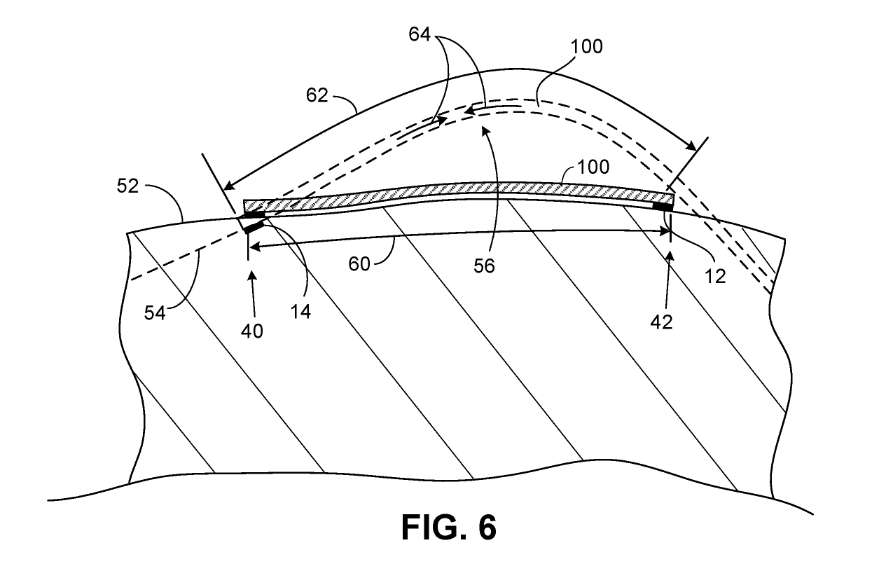

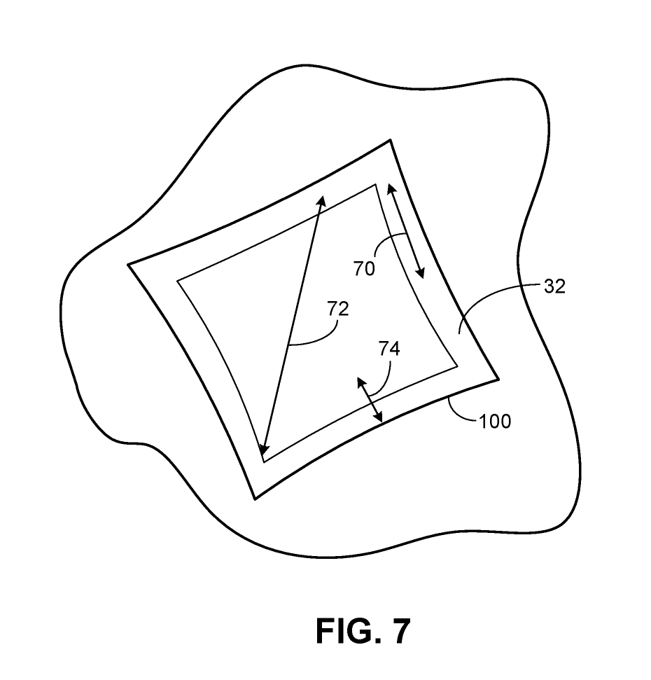

FIG. 6 is a sectional view of a part of a body and a barrier in two different states.

FIG. 7 is a view of a barrier on skin.

One example of a product used to protect a vulnerable exposed feature temporarily, for example, during a shower, is the HydroSeal™ product distributed by 2G Medical, LLC, of Clearwater, Fla.

As shown in FIG. 1, the HydroSeal product 8 includes a square (or otherwise rectangular) transparent plastic membrane 10. In typical implementations of the existing product 8, the membrane 10 is formed of a relatively un-stretchable plastic membrane. Arranged along the four edges at the periphery of the plastic membrane 10 are four long rectangular paper release tabs 12, 14, 16, 18. Each of the paper release tabs 12, 14 on opposite sides of the product has a length 20 that is shorter than the length of the corresponding side of the product where the tab is located. Each of the paper release tabs 16, 18 on the remaining two opposite sides of the product has a length 22 that is the same as the length of the corresponding side of the product where the tab is located. At each of the corners of the product one end of one of the paper release tabs 16, 18 overlies a corresponding end of one of the paper release tabs 12, 14. For example, the end 24 of the paper release tab 14 is sandwiched between the end 26 of paper release tab 16 and a surface 30 of the plastic membrane 10.

As shown in more detail in FIG. 2 (taken at section 2-2 of FIG. 1) the paper release tab 14 (including its end 24) has one surface 34 attached to the surface 30 of the membrane 10 by a thin coating of adhesive 32. The surface 34 of the paper release tab 40 has a glossy finish that enables the paper release tab to be held by the adhesive 32 until the product is to be used, and then to be released easily from the adhesive 32 by peeling the tab away from the adhesive. Similarly, the outer portion 36 of the end 26 of paper release tab 16 has its surface 38 (which has a glossy finish) attached to the surface 30 of the membrane 10 by the adhesive 32.

The coating of adhesive 32 extends continuously without interruption around the perimeter along all four sides of the membrane 10 and has a constant width. A variety of adhesives could be used. The adhesive therefore surrounds a central area of the membrane that is free of adhesive. The two paper release tabs 12, 14 have essentially the same width as the width of the adhesive coating. The other two paper release tabs 16, 18 are wider than the width of the adhesive coating.

As a result, as illustrated in FIG. 1, each of the paper release tabs 16, 18 has a strip 28 spaced away from the peripheral edge of membrane 10 that does not have a surface attached by adhesive to the membrane 10. As shown in FIG. 3 (taken at section 3-3 of FIG. 1), the paper release tab 16 includes both the stripe 28 that does not touch the adhesive coating 32 and a remaining stripe 29 that does adhere to the adhesive coating 32. A user's finger can therefore easily be slipped between the strip 28 and the surface of the membrane.

Therefore, before the product is placed into use, it includes a flat flexible transparent rectangular plastic membrane 10 and four paper release strips temporarily attached to the adhesive coating along the four sides of the membrane 10 with the ends of release strips 16, 18 overlying the ends of release strips 12, 14.

As shown in FIG. 4, to place the product into use, say to protect a stitched incision on a knee (a vulnerable exposed feature), a person can use her finger to peel paper release strips 12 and 14 from the adhesive coating and then press the exposed adhesive coating of the membrane onto the skin along two lines 40, 42 that are spaced apart and lie on opposite sides of an exposed feature 44. The outside surface of the membrane 10 opposite the surface that bears the adhesive coating faces away from the person's skin and the paper release strips 16, 18 lie between the membrane 10 and the skin, that is, underneath the membrane 10. The person then can use her finger to reach in between the skin and the membrane and then under the un-adhered strips 28, 29 to easily pull the adhered border away from the membrane and then press the exposed adhesive against the skin at two other locations 46, 48. The adhesive is chosen to provide a temporary secure seal against the passage of environmental materials from outside the product into the space between the membrane and the skin. This leaves membrane 10 (without the paper release strips) sealed to the person's skin along a continuous closed band of attachment that surrounds the exposed feature 44 and seals the space that is in the vicinity of the exposed feature 44 and is between the membrane 10 and the skin against exposure to environmental materials.

In some examples, the product can be attached to a person just before taking a shower in order to shield the exposed feature from water. Later, for example after the shower, the membrane can be peeled away from the skin and discarded. The adhesive is chosen to release from the skin without damaging the skin when the person peels the product off, after use.

As shown in FIG. 5, in some uses, the exposed feature is located in a place such that when the membrane is attached to the skin to shield the exposed feature, as explained earlier, the motion (for example, bending) of one part of the body (for example, the calf or forearm) to which one side of the membrane is attached relative to another part of the body (for example, the thigh or upper arm) causes tension such as tension 50 on the membrane (in this case along the length of the leg or arm). The tension can impart a force to the adhesive seal (for example on the side attached to the thigh or upper arm) causing the membrane to peel away from the skin, breaking the seal and increasing the risk of exposure of the vulnerable exposed feature to an environmental material, e.g., water.

Improved Barrier

As shown in FIG. 6, a significantly better barrier can be formed by replacing the relatively non-stretchable membrane described above with a relatively stretchable resilient membrane 100 shown in two successive orientations during use (such as when an elbow is in an extended position and then in a contracted position). When a portion 60 of the body moves, changes its position, or changes its orientation in such a way as to change the surface contour 52 of the skin or another exposed feature, a force 56 can be imposed on the surface of the membrane 100 facing the contour 52. As the force 56 is imposed, the contour of the exposed feature changes from contour 52 to contour 54 (shown in dashed line), and the length or width 60 between the adhered locations 40, 42 can increase from length 60 to a greater length or width 62 because the membrane 100 is stretchable. This can occur without detaching the membrane from the skin along the seal, for example, at locations 40, 42. Although the force 56 creates a tension 64 parallel to the surface of the membrane 10 tending to pull the sides of the membrane at locations 40, 42 away from the skin, as long as the forces imparted by the tension are less than the adherent forces of the adhesive, the forces imparted by the tension will tend to cause the membrane 10 to stretch to absorb the force while the seal remains intact.

If the force 56 is removed and the contour 54 tends to return to the original contour 52, the resilience of the material of the membrane 100 will enable it to tend to return to its original shape and dimensions.

As shown in FIG. 7, forces tending to pull on the adhered sides of the membrane 100 can be caused in many ways and have a variety of directions and magnitudes. The forces can be caused by flexing of joints, by twisting or rotating of body parts, by contraction and relaxation of muscles, and in other ways. The resulting forces can extend diagonally 72, longitudinally 70 along one side of the membrane 100, perpendicularly 74 to one of the sides, and in other directions, and can result from a variety of causes at a given time.

The material for the membrane 100 can be selected to impart any one or combinations of two or more of the following characteristics:

1. The membrane can be flexible enough to permit easy attachment to the person and easy removal. The flexibility can be sufficient to accommodate motion of the skin or other parts of the body in the vicinity of the exposed feature being shielded.

2. The membrane can be transparent to permit easy viewing of the condition of the exposed feature and the skin in the vicinity of the exposed feature. Transparency also makes it easy to confirm that the membrane has been applied in such a way that the surrounding seal provided by the adhesive against the skin is not touching the exposed feature and to make it easy to observe the exposed feature. The membrane can be left free of any printed information so as to provide an unobstructed view. In some implementations, color dies can be incorporated into the material of the membrane for various purposes including color coding the uses of or locations where the membrane can be applied most effectively.

3. The membrane can be formed of one or more distinct layers that can impart different characteristics to the membrane. For example, stretchability could be imparted to one layer and resilience to another layer so that in combination the two layers form a membrane that is both stretchable and resilient. In some implementations, a layer facing the skin can have properties that are suitable for touching the skin or the exposed feature. For example, that layer may be hypoallergenic and formed of a material that does not naturally adhere to the skin. Another layer facing the ambient environment may have qualities that enable it to shed environmental materials of certain kinds or qualities that enable it to attract or adhere to environmental materials. For example, this other layer could be oleophilic, oleophobic, hydrophobic, or hydrophilic among other things.

4. An important characteristic is that the membrane or the layers of which it is formed enable the membrane to be stretchable to accommodate adverse forces of the kind described above. A range of stretchabilities (e.g. elongation at break) can be used. The stretchability may be within the range of 150% to 1000%, or in particular within the range of 300% to 800%, or in a specific range from 500 to 700%. In some implementations, the stretchability could be approximately 660%.

5. In typical applications, the membrane or at least one of the layers of which it is formed will be impenetrable by (i.e. will shield against) particular environmental materials, such as water or oil. In some cases the membrane will be impenetrable by liquids, but will be penetrable by air or particular gases.

6. The membrane can be formed of a polymeric material, e.g., a polyurethane such as an aromatic polyether polyurethane (for example, the aromatic polyether polyurethane available commercially as product MT-2001 from American Polyfilm, Inc., in Branford, Conn.). Other materials may also be suitable including co-polyester films. The published properties of the MT-2001 product include the following, and films having properties within effective ranges above and below the indicated values would also be useful. Films that exhibit good resilience, good low temperature flexibility, excellent hydrolytic stability and resistance to attack by microorganisms, weldability or combinations of such qualities would be useful.

| Properties | Test Method | Values | |
|---|---|---|---|
| Durometer | ASTM D2240 | 82 Shore A | |
| Specific Gravity | ASTM D792 | 1.1 | |
| Elongation | ASTM D412 | 660% | |
| at Break Elast | | | |
| Tensile Strength | ASTM D412 | 5,080 | psi |
| at Break Elast | | | |
| 100% Modulus | ASTM D412 | 725 | psi |
| 300% Modulus | ASTM D412 | 1,100 | psi |
| Tear Strength | ASTM D624 Die C | 430 | pli |
| Abrasion Loss | ASTM DIN 53.516 | 20 | mm³ |

| | |
|---|---|
| Melt Range | 320-340° F. (160-170° C.) |

7. The membrane can be formed of a material that is non-irritating to the skin of the person and does not inherently adhere to the skin.

8. The membrane can include a hydrolytically stable material so that water does not significantly affect its useful properties.

9. The membrane can have a thickness that is between 2 mils and 4 mils, for example.

In general, in selecting an appropriate material for the membrane it can be useful to for the membrane to be not only stretchable but also resilient enough so that as the person using the product continues to move parts of her body in the vicinity of the product the membrane can stretch, contract, stretch, and contract repeatedly without breaking. In general, using a membrane that is relatively thinner will tend to enable a greater stretchability but the membrane should not be so thin that it can be easily broken by motion of the user's body.

The product can have a wide variety of configurations including the following:

1. The product can be rectangular, square, round, or any other simple geometric shape, and can be of a more complex shape suited to a particular part of the person where the exposed feature is located.

2. The product can have a variety of sizes and aspect ratios including 5″×5, 7″×7″, 9″×9″, 10″×12″, 12″×14″, or 7″×22″. The sizes and aspect ratios can be selected in particular to take advantage of the stretchability of the material of the membrane and to be suited to the part of the person where the exposed feature is located.

4. The stretchability of the product can be achieved not only by the stretchability of the material of the membrane itself, but also by other features associated with the material including gussets, folds, accordions, resilient supplemental devices and structures attached to the material, and in other ways.

5. The paper release strips can take on a wide variety of shapes and sizes and materials and be provided with tabs and other mechanisms to permit them to be easily removed from the membrane while at the same time permitting easy attachment of the membrane for the skin. The release strips can be made of materials other than paper, such as plastic or foil or combinations of them. Other arrangements of release strips can be provided to enable a person to reach under a partially attached membrane to remove remaining release strips. There can be more than one strip per side of the product and one strip can serve more than one side. Other approaches to forming the seal may also be possible without using release stress. The release strips can be any kind of sheet material that bears a release coating such as a silicone coating or any other coating that enables easy release from the adhesive.

Other implementations are within the scope of the following claims.

1. A barrier comprising
a water impenetrable membrane having two sides and a perimeter,
on one side of the membrane, a continuous strip of adhesive configured to be adhered to skin of a person to form a water-tight seal with the skin along an entire perimeter of the membrane, the water-tight seal surrounding an adhesive-free central area of the membrane, the adhesive-free central area of the membrane configured to not be adhered to the skin,
the adhesive-free central area of the membrane being stretchable between a first, un-stretched configuration and a second stretched configuration without breaking the water-tight seal along the entire perimeter of the membrane, the adhesive-free central area of the membrane configured to remain un-adhered to the skin,
the stretched configuration being elongated by at least 150% relative to the un-stretched configuration.

2. The apparatus of claim 1 in which the membrane comprises a polymeric material.

3. The apparatus of claim 2 in which the polymeric material comprises a polyurethane.

4. The apparatus of claim 3 in which the polyurethane comprises an aromatic polyether polyurethane.

5. The apparatus of claim 1 in which the membrane is transparent.

6. The apparatus of claim 2 in which the membrane has a thickness in the range of 2 mils to 4 mils.

7. The apparatus of claim 1 in which the continuous strip of adhesive has adhesive properties to seal the membrane to skin temporarily and to permit removal of the membrane without damaging the skin.

8. A method comprising
attaching a barrier to skin of a person to form a seal between the barrier and the skin continuously along a strip that surrounds an exposed feature of the person, to shield the exposed feature temporarily against exposure to an environmental material, and
after the barrier has been attached, stretching the barrier to accommodate a tension imparted to the barrier by motion of a part of the person in the vicinity of the exposed feature, to prevent the tension from breaking the seal.

9. The method of claim 8 in which forming the seal between the barrier and the skin comprises pressing an adhesive material against the skin.

10. The method of claim 8 comprising breaking the seal and removing the barrier from the skin.

11. The method of claim 8 comprising contracting the barrier when the tension imparted to the barrier is released.
